(19) United States
(12) Patent Application Publication
(10) Pub. No.: US 2019/0324023 A1
(43) Pub. Date: Oct. 24, 2019

(54) DETECTION OF EXTRACELLULAR VESICLES USING NANOPARTICLES

(71) Applicants: IMRA America, Inc., Ann Arbor, MI (US); JAPANESE FOUNDATION FOR CANCER RESEARCH, Tokyo (JP)

(72) Inventors: Yuki Ichikawa, Ann Arbor, MI (US); Kiyotaka Shiba, Tokyo (JP); Sachiko Matsumura, Tokyo (JP)

(21) Appl. No.: 15/958,241

(22) Filed: Apr. 20, 2018

(51) Int. Cl. G01N 33/543

(52) U.S. Cl. CPC G01N 33/54306

(57) ABSTRACT

The present disclosure provides examples of methods and kits for easily detecting, classifying and/or purifying extracellular vesicles. The method can include subjecting, to a density gradient centrifugation, a sample solution in which the extracellular vesicles and nanoparticles coated with ligand that specifically binds to molecule present on the surface of the extracellular vesicles are mixed.

### BACKGROUND

### Field

The present disclosure relates to a simple method for separating and detecting extracellular vesicles. In particular, the present disclosure relates to a method for classifying exosomes into subclasses and detecting the exosomes classified into the subclasses.

### Description of the Related Art

Extracellular vesicles are nanometer-sized vesicles released from cells, and are classified into exosomes, microvesicles and apoptotic bodies, according to their cellular origin. Among them, exosomes, which may be possibly utilized for diagnosis and treatment, have been actively researched in recent years.

Exosomes are cell-derived vesicles of approximately 100 nm in diameter which are present on the outside of cells, and they are found in large amounts in any body fluids such as blood, saliva and urine. Exosomes have lipids and proteins derived from cell membranes on their surface and have nucleic acids such as mRNA and miRNA and proteins therein, and thus contain information derived from the cells which have released them. Therefore, the information included in exosomes can be utilized for a diagnostic marker as a biomarker.

It is also known that biological information molecules having these nucleic acids and proteins function in cells having exosomes incorporated therein, and it has been shown that intercellular communication by exchange of nucleic acids and proteins occurs through exosomes. Therefore, it has been thought that exosomes can be utilized not only for the diagnosis but also in the field of prevention and treatment (He, C et al., 2018, Theranostics, vol. 8(1), p. 237-255).

Although the biological significance of exosomes has been clarified as described above, consensus about the definition of exosomes itself has not been obtained among researchers. How exosomes having a high diversity are to be classified and defined has been an issue largely related to future exosome research.

### SUMMARY

The present disclosure provides various methods for classifying extracellular vesicles into subclasses according to their surface components and detecting the extracellular vesicles classified into the subclasses. For example, the methods include detecting an exosome subclass having a particular constituent utilizing an antigen present on the surface of the exosome or a membrane constituent of the exosome by precipitating.

Embodiments of the disclosure include a method for detecting, fractionating and/or purifying extracellular vesicles, and a kit using the same.

In an embodiment, a method for detecting, fractionating and/or purifying extracellular vesicles, comprises preparing nanoparticles coated with a ligand that specifically binds to a molecule present on the surface of the extracellular vesicles to be detected, and a sample solution in which the nanoparticles and the extracellular vesicles are mixed; providing at least one medium layer with a density higher than that of the sample solution; overlaying the sample solution on the at least one medium layer; and subjecting the at least one medium layer overlaid with the sample solution to low-speed centrifugation.

In an embodiment, a kit for detecting, classifying and/or purifying extracellular vesicles, comprises nanoparticles coated with a ligand that specifically binds to a molecule present on the surface of the extracellular vesicles to be detected; and a density gradient solution.

The foregoing summary and the following drawings and detailed description are intended to illustrate non-limiting examples but not to limit the disclosure.

### BRIEF DESCRIPTION OF THE DRAWINGS

FIG. 1 is a schematic view illustrating various embodiments in which extracellular vesicles are sedimented by density gradient centrifugation;

FIG. 2 is an absorption spectrum of an example of an MFGE8-modified gold nanoparticle colloidal solution;

FIG. 3 is an absorption spectrum of an example of an MFGE8-modified gold-platinum alloy nanoparticle colloidal solution;

FIG. 4 is an absorption spectrum of an example of an anti-CD81 monoclonal antibody-modified gold nanoparticle colloidal solution;

FIG. 5 is an image showing the results of Western blot analysis (left) of the expressed protein in each fraction of exosome samples prepared by density gradient centrifugation from the culture medium of human pancreatic cancer cell line, MiaPaca-2, and a graph showing the number of particles in each fraction (right);

FIG. 6A is a photograph showing an example mixture of MFGE8-modified gold nanoparticles and PBS or exosomes in fractions 2 or 8, or a mixture of BSA-modified gold nanoparticles and exosomes in fraction 3; and FIG. 6B is a photograph of each fraction after density gradient centrifugation, and a schematic view illustrating the manner in which exosomes and modified gold nanoparticles were present;

FIG. 7A includes examples of images obtained by adding biotinylated annexin V to exosome fraction 1 (left) and exosome fraction 3 (right), labeling phosphatidylserine on the exosome membrane surface with anti-biotin antibody conjugated gold particles and observing with AFM; and FIG. 7B is a graph showing the proportion of exosomes bound to gold nanoparticles in the examples of FIG. 7A;

FIG. 8 are example AFM images showing that the binding between exosomes and MFGE8-modified gold nanoparticles can be inhibited by addition of recombinant MFGE8;

FIG. 9 includes example photographs showing that density gradient centrifugation was performed using MFGE8-modified alloy nanoparticles and exosomes contained in each fraction sedimented;

FIG. 10 is a photograph of example experimental results showing that the amount of sedimentation by density gradient centrifugation was dependent on the number of exosome particles;

FIG. 11 is a diagram of example experimental results showing that when MFGE8 was added to an exosome fraction, MFGE8-modified alloy nanoparticles did not sediment;

FIG. 12A is a diagram showing an example of the detection of exosomes using anti-CD81 antibody-modified gold nanoparticles; and FIG. 12B is a diagram showing an example of the detection of exosomes by two layers with different densities;

FIG. 13A is a diagram showing example results of studying the densities of the medium layers; and FIG. 13B is diagram also showing example results of studying the densities of the medium layers; and

FIG. 14 is a diagram showing example results of the detection using unfractionated exosomes.

The figures depict various embodiments of the present disclosure for purposes of illustration and are not intended to be limiting. Wherever practicable, similar or like reference numbers or reference labels may be used in the figures and may indicate similar or like functionality.

### DETAILED DESCRIPTION

In order to use exosomes occurring as heterogeneous populations for diagnosis and treatment, it may be advantageous to classify exosomes into subclasses. If exosomes can be classified into subclasses, it is thought that biomarkers can be concentrated from particular cells, and it is thus possible to perform diagnosis with higher accuracy. For use in treatment, it also may be advantageous to concentrate exosomes contributing to the treatment as a tool of cellular communication. Accordingly, attempts have been made to classify exosomes into subclasses according to the antigens exposed on their surfaces, their densities, and the like.

Methods for purifying extracellular vesicles include a method for purifying them using a kit with reagents and a method for purifying them by centrifugation depending on their densities. However, the kits often contain no description of the separation principle and the details of reagents. Therefore, it may be unclear whether all of the desired extracellular vesicles can have been recovered. Since methods using ultracentrifugation such as density gradient centrifugation are time-consuming and labor-intensive, simpler methods described below are provided.

The present disclosure describes a method for purifying particular extracellular vesicles comprising: modifying nanoparticles with molecules capable of adsorbing the extracellular vesicles; and causing a plurality of nanoparticles to aggregate via extracellular vesicles to sediment aggregates. As shown in the following Examples, an extracellular vesicle having a particular surface marker can be sedimented at low-speed centrifugation, so that extracellular vesicles can be very easily fractionated. Hereinafter, exosomes will be mainly described as extracellular vesicles. However, extracellular vesicles other than exosomes, classified as microvesicles or apoptotic bodies can be detected, classified into subclasses and purified in the same manner as the exosomes.

As shown in the Examples, nanoparticles function as an anchor to sediment extracellular vesicles including exosomes and also function as an indicator for visually indicating the sedimentation state of the extracellular vesicles. For functioning as an anchor, it can be preferable to use materials with high density, and it is better to use materials containing a sixth periodic element of the periodic table. Examples include gold (19.32 g/cm³) or platinum (21.45 g/cm³) in an amount of 50% by weight or more and 100% by weight or less. Among sixth periodic elements, tantalum (Ta), tungsten (W), rhenium (Re), osmium (Os), iridium (Ir), platinum (Pt) and gold (Au) can be used due to their high density exceeding 15 g/cm³. The materials for fine particles may be composed of a single element, but may be composed of a plurality of elements such as an alloy, solid solution or a compound such as an oxide.

As an indicator for visually indicating the presence of exosomes, light absorption by plasmon resonance of a noble metal such as gold or platinum can be utilized. Since gold nanoparticles exhibit a red color and nanoparticles with gold-platinum core-shell structure exhibit a black color, sedimentation of nanoparticles can be clearly confirmed visually.

It is also known that new plasmon mode appears by causing a plurality of noble metal nanoparticles having plasmon resonance to aggregate, and the light absorption spectrum, that is, known for color changes (Tatsumoto, E. et al. 2012, the Second Meeting in Heisei 24, Kansai Analysis Sciety; Cha, H. et al., 2014, ACS Nano, vol. 8, No. 8, p. 8554-8563). It is also possible to use fine particles composed of such nanoparticle aggregates. It is possible to perform detection and quantification not only by visual indicators but also by producing alloy nanoparticles with magnetic metal such as iron to perform detection and quantification with magnetism.

The fine particles may be composed of a single particle, but may be clusters composed of a plurality of particles. The fine particle refers to particle having a diameter of 1 nm to 10 μm, and a nanometer sized particle, nanoparticle, is preferably used. The size of nanoparticles in the longest axis may be 500 nm or less and 5 nm or more, preferably 200 nm or less and 20 nm or more, and most preferably 100 nm or less and 30 nm or more. Depending on the density of the materials, if the size is too large, the fine particles themselves sediment, whereas if it is too small, sedimentation by centrifugation requires a long time or a very high rotation speed.

As shown in the following Examples, embodiments of methods include: modifying nanoparticles with molecules capable of binding to molecules present on the surface of extracellular vesicles; causing the nanoparticles to bind to the molecules present on the surface of the extracellular vesicles; and by utilizing formation of aggregates of the nanoparticles and the extracellular vesicles due to binding, precipitating the formed aggregates via the low-speed centrifugation by density gradient centrifugation. The density gradient may be a continuous density gradient or a step density gradient.

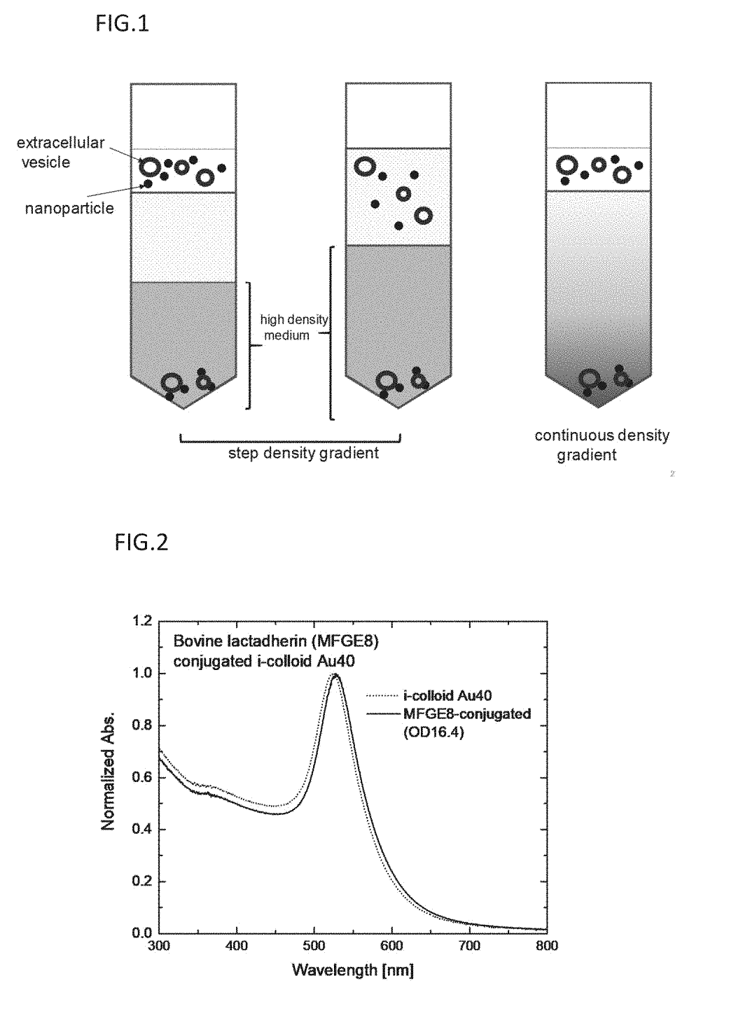

FIG. 1 is a schematic view illustrating various embodiments in which extracellular vesicles are sedimented by density gradient centrifugation. The nanoparticles are modified with molecules capable of specifically binding to particular molecules on the surface of extracellular vesicles. Among the extracellular vesicles, those having particular molecules exposed on their surfaces bind to the nanoparticles. By setting an appropriate density gradient and centrifugal condition, the present inventors have found the condition in which extracellular vesicles bound to nanoparticles can be distinguished from extracellular vesicles not bound to nanoparticles and only the extracellular vesicles bound to nanoparticles can be sedimented.

The density gradient may be a step density gradient or a continuous density gradient. The step density gradient can be obtained by layering several media with different densities and then layering thereon a mixture of nanoparticles and a sample containing extracellular vesicles (FIG. 1, left), or by mixing the nanoparticles and the sample containing the extracellular vesicles with a medium with appropriate density and directly layering on a medium with high density (FIG. 1, middle). FIG. 1, left schematically illustrates a case in which a mixture of the nanoparticles and the samples is layered on two media with different densities, but the density gradient layers are not limited to two layers and any number of layers may be provided as necessary.

For the density of the medium, the density of the high density layer is a factor under which only the nanoparticles bound to extracellular vesicles are sedimented and the nanoparticles not bound to extracellular vesicles are not sedimented. The high density layer varies depending on the type and size of the particles to be used, but when using nanoparticles of 100 nm or less and 30 nm or more in the longest axis comprising a noble metal with a density exceeding 15 g/cm³, the high density layer have a density of 1.053 g/mL (10% in the case of iodixanol) or more and 1.32 g/mL (60% in the case of iodixanol) or less. The above-mentioned density is a density when iodixanol is mixed with pure water. When mixing iodixanol with a buffer solution such as physiological saline, the density can be about 0.5% higher than the above-mentioned value. Solutes other than iodixanol whose density can be set at the same level as iodixanol may be also used.

In the following Example, the density gradient is created by iodixanol, but it can be created by using any compound conventionally used in density gradient centrifugation, including salts such as cesium chloride or sodium bromide; sugars such as sucrose, sorbitol or glycerol; triiodobenzene-based compounds such as Metrizamide or Nycodenz; polymer-based compounds such as Ficoll; and colloidal silica-based compounds such as Percoll.

The centrifugation speed and centrifugation time also can vary depending on the density of the medium to be used, but considering that examination of the sample may be performed promptly after centrifugation, sedimentation can be performed in a centrifugation time of 30 minutes or less, preferably 10 minutes or less and more preferably 5 minutes or less. The centrifugation speed for precipitating extracellular vesicles bound to nanoparticles in a short time can be set in the range of 1 g to 2000 g, preferably 200 g to 1500 g, more preferably 500 g to 1,500 g.

The method for producing fine particles may be either a chemical method or a physical method. However, it has been reported that since the fine particles prepared by the in-liquid laser ablation method used in the following Examples contain neither a surfactant nor a reaction by-product in a colloidal solution, the surfaces of the fine particles are chemically-bare and in particular, ligand molecules are passively adsorbed the surface with high efficiency (Cederquist, K. B. et al., 2017, Colloids and Surface B: Biointerfaces, vol. 149, p. 351-357). Therefore, the in-liquid laser ablation is a particularly preferable production method in some embodiments.

The surface of the nanoparticle is modified with a ligand that specifically binds to a molecule present on the extracellular vesicle. As described above, since the nanoparticles bind to the extracellular vesicles via the ligand on the surface of the nanoparticles and sediments, the extracellular vesicles binding to the ligand can be fractionated and purified. Since the extracellular vesicles binding to particular ligands can be selectively sedimented, extracellular vesicles can be classified into subclasses by the binding to ligands.

As molecules exposed on the surface of extracellular vesicles, for example, on an exosome which is one of extracellular vesicles, various marker molecules are known such as CD9, CD13, CD31, CD44, CD63, CD81, Rab5b, MHC, α2-macrogloblin, LAMP1/2, ICAM-1, Flotilin 1, PSMA, Tetraspanin-1, SLC44A4, PROM2, CD133, CD14, LRRC26, integrin, ceramide, cholesterol, phosphatidylserine, EpCAM and sugar chains. Also, researches on exosomes may proceed and new surface markers may be found. Exosome surface markers that are newly found and surface markers other than the above mentioned surface markers can be utilized. Although examples of the surface markers for exosomes are described here, for other extracellular vesicles such as microvesicles and apoptotic bodies, molecules that specifically bind to marker molecules such as proteins, lipids and sugar chains can be used.

Molecules (ligands) that function to specifically bind to target surface markers include antibodies, proteins, peptides and nucleic acids. Examples of the antibodies specific to the surface markers for exosomes includes antibodies to CD9, CD63 and CD81, known as so-called general exosome markers, and antibodies to proteins exposed on the surfaces of the exosomes. Examples of the proteins include annexin V and Lactadherin (or milk fat globule-EGF factor 8 protein, MFGE8) which specifically bind to phosphatidylserine, and lectins which bind to sugar chains. Examples of the peptides include a peptide aptamer that binds to EpCAM, a protein present on the surface of the exosome (Yoshida, M et al. 2017, Biotechnol. Bioeng. doi: 10.1002/bit.26489). Examples of the nucleic acids include a single-stranded DNA aptamer that binds to CD63 (Jiang, Y et al., 2017, Angew. Chem. Int. Ed. vol. 56, 11916-11920).

For the binding between the fine particle and the ligand molecule, a method by chemical bonding can be utilized in addition to or as an alternative to the above-mentioned physical adsorption. For example, ligand molecules having thiol (—SH) or disulfide (—S—S—) can be chemically bound to the surface of metals such as gold. Instead of directly binding molecules that function to specifically bind to target molecules on extracellular vesicles to the surfaces of the nanoparticles, it is also possible to bind the molecules via linkers to the surfaces of the nanoparticles.

In some embodiments, it may be desirable to perform blocking of the surfaces of the nanoparticles not bound by the ligands in order to reduce or avoid nonspecific binding of nontarget molecules on the surfaces of extracellular vesicles. Examples of the blocking molecules include proteins such as BSA, surfactants such as polysorbate 80 (Tween-80) or polysorbate 20 (Tween-20), polymers such as polyvinylpyrrolidone (PVP), and mixtures thereof. Blocking may be performed after coating the nanoparticles with the ligands or before use. Blocking may be also performed using a buffer solution containing the above-mentioned blocking molecule as a solution for storing the nanoparticles.

The following Examples are intended to illustrate, but not to limit, aspects of the technology.

### 1. Production of Nanoparticle Coated with Ligand

### Example 1

### Preparation of MFGE8 (Milk Fat Globule-EGF Factor 8 Protein, Lactadherin)-Modified Gold Nanoparticle

A colloidal solution of a laser-fabricated gold nanoparticle, i-colloid Au 40 nm (Product No. icAu 40-1-100, manufactured by IMRA America, Inc.; zeta potential <−60 mV; colloid conductivity <10 μS/cm), having an average particle diameter of approximately 40 nm and OD of 1 at its plasmon peak wavelength, was prepared. The colloidal solution of 14.1 mL was placed in a 15 mL centrifuge tube, 0.6 mL of a 0.1 M borate buffer solution (pH 8.2) is added thereto, and the mixture was stirred.

In another 15 mL centrifuge tube is placed 0.1 mL of deionized water and 0.1 mL of a 0.5 mg/mL Bovine Lactadherin (MFGE8) solution (manufactured by Haematologic Technologies, Inc.), and 4.8 mL of the above-prepared mixture of the borate buffer solution (pH 8.2) and the gold nanoparticle colloidal solution is poured thereinto. The mixture was stirred with a vortex mixer, and incubated on a shaker at room temperature at 100 rpm for 30 minutes. MFGE8 is a protein that binds to phosphatidylserine (PS) on the surfaces of exosomes.

The reaction mixture was subjected to centrifugation at 4,000 g for 30 minutes at room temperature to sediment the gold nanoparticles. The supernatant was removed, 0.3 mL of a 4 mM borate buffer solution (pH 8.2) was added to the precipitate, and the mixture was stirred with a vortex mixer to redisperse the gold nanoparticle.

The absorption spectrum of the MFGE8-modified gold nanoparticle colloidal solution thus prepared was measured with an ultraviolet-visible spectrophotometer (UV-2700, manufactured by Shimadzu Corporation) and normalized with its plasmon absorption peak wavelength, and compared with the absorption spectrum of an unmodified gold nanoparticle colloidal solution. The results are shown in FIG. 2. The plasmon absorption peak wavelength of the MFGE8-modified gold nanoparticle colloidal solution was 528 nm and its OD was 16.4. The prepared MFGE8-modified gold nanoparticle colloidal solution was stored at 4° C. for use.

### Example 2

### Preparation of an MFGE8-Modified Gold-Platinum Alloy Nanoparticle

A colloidal solution of a laser-fabricated gold-platinum alloy nanoparticle, i-colloid AuPt 40 nm (Product No. icAuPt 40-1-100, manufactured by IMRA America, Inc.; zeta potential <−50 mV; colloid conductivity <10 μS/cm), having an average particle diameter of approximately 40 nm and OD of 1 at a wavelength of 400 nm is prepared. The colloidal solution of 14.1 mL was placed in a 15 mL centrifuge tube, 0.6 mL of a 0.1 M borate buffer solution (pH 8.2) was added thereto, and the mixture was stirred.

In a 1.7 mL centrifuge tube was placed 0.1 mL of a 0.5 mg/mL MFGE8 solution, and 0.9 mL of the above-prepared mixture of the borate buffer solution (pH 8.2) and the gold-platinum alloy nanoparticle colloidal solution was poured thereinto. The mixture was stirred with a vortex mixer, incubated on a shaker at 200 rpm for two hours at room temperature, and then allowed to stand at 4° C. overnight.

The reaction mixture contained in the tube was subjected to centrifugation at 4,000 g for 30 minutes at room temperature to sediment gold-platinum alloy nanoparticles. The supernatant was removed to leave a pellet on the bottom of the tube. To the tube was added 0.5 mL of a 4 mM borate buffer solution (pH 8.2), and the mixture was stirred with a vortex mixer to redisperse the gold-platinum alloy nanoparticles.

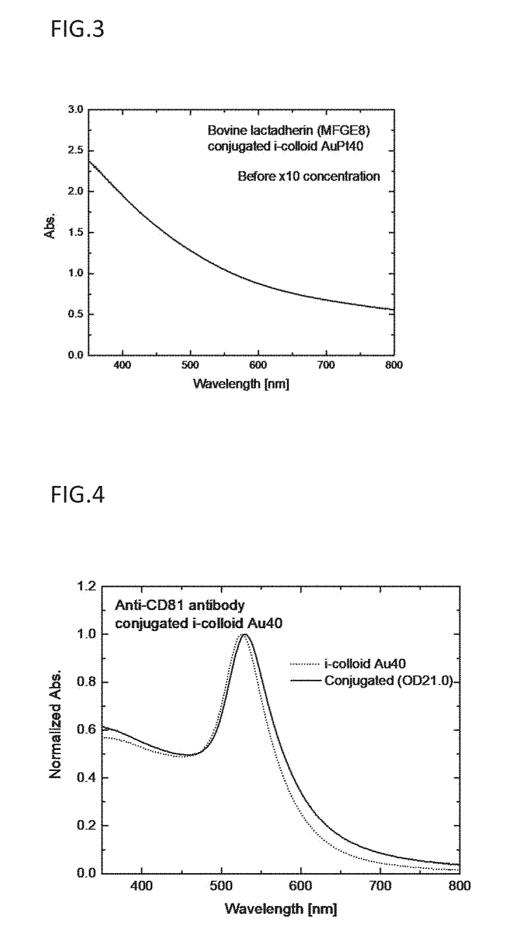

The absorption spectrum of the MFGE8-modified gold-platinum alloy nanoparticles (40 nm) thus prepared was measured in the same manner as in Example 1. The results are shown in FIG. 3. The OD was 1.96 at a wavelength of 400 nm.

In addition, the particle diameter of the nanoparticle was measured by a dynamic light scattering method (Zetasizer Nano ZS-90, manufactured by Malvern Instruments Ltd.). The measured hydrodynamic diameter was 74.61 nm, which was confirmed to have increased by approximately 19 nm in size compared with the hydrodynamic diameter of 55.44 nm before MFGE8 modification.

One mL of the above-prepared MFGE8-modified gold-platinum alloy nanoparticle (40 nm) colloidal solution was subjected to centrifugation at 4,000 g for 30 minutes at room temperature, 0.9 mL of the supernatant was removed to concentrate the MFGE8-modified gold-platinum alloy nanoparticles by 10 times, and the concentrate was stored at 4° C. until use.

### Example 3

### Preparation of Anti-CD81 Monoclonal Antibody-Modified Gold Nanoparticle Colloidal Solution

The gold nanoparticle colloidal solution used in Example 1 was prepared and 14.1 mL of the solution was placed in a 15 mL centrifuge tube, 0.6 mL of a 0.1 M phosphate buffer solution (pH 7.0) was added thereto, stirred with a vortex mixer, and allowed to stand.

Two hundred μL of an anti-CD81 monoclonal antibody solution (mouse monoclonal CD 81, M 38 (IgG1), manufactured by EXBIO Praha, a.s.) and 50 μL of a 0.1 M phosphate buffer solution were placed in a 1.5 mL centrifuge tube, then mixed.

In another 1.5 mL centrifuge tube was placed 20 μL of the anti-CD81 monoclonal antibody-phosphate buffer solution thus prepared, and 980 μL of the above-prepared mixture of the phosphate buffer (pH 7.0) and the gold nanoparticle colloidal solution was poured thereinto. The mixture was stirred with a vortex mixer, and incubated on a rotary shaker at room temperature for two hours.

Then, the half volume, 0.5 mL out of 1.0 mL of the above mixed solution was taken out, and placed in a new 1.5 mL centrifuge tube. 0.5 mL of 4 mM phosphate buffer solution (pH 7.0) containing 10 mg/mL of BSA was added thereto, stirred with a vortex mixer, and incubated overnight at 4° C. After incubated, the mixture was stirred again with a vortex mixer, subjected to centrifugation at 2,500 g for 15 minutes at room temperature to sediment gold nanoparticles. Approximately 0.95 mL of the supernatant was removed to leave a pellet on the bottom of the tube.

The absorption spectrum of the anti-CD81 monoclonal antibody-modified gold nanoparticle colloidal solution thus prepared was adjusted by dilution with a 4 mM phosphate buffer solution (pH 7.0) containing 5 mg/mL of BSA so that the OD is approximately 20 at the final plasmon absorption peak wavelength as measured in the same manner as in Example 1.

The absorption spectrum measured with an ultraviolet-visible spectrophotometer was normalized with the plasmon absorption peak wavelength, and compared with the absorption spectrum of an unmodified gold nanoparticle colloidal solution. The results are shown in FIG. 4. The plasmon absorption peak wavelength of the anti-CD81 monoclonal antibody-modified gold nanoparticle colloidal solution was 530 nm and its OD was 21.0. The prepared anti-CD81 monoclonal antibody-modified gold nanoparticle colloidal solution was stored at 4° C. 2. Detection of exosome with nanoparticle

### Example 4

### Detection of Exosome Using MFGE8-Modified Gold Nanoparticle

Using the MFGE8-modified gold nanoparticles (40 nm) prepared in Example 1 (hereinafter referred to as M40), exosomes were detected utilizing the binding between MFGE8 and phosphatidylserine on the surface of the exosomes.

An exosome sample was prepared from a culture medium of MiaPaca-2 human pancreatic cancer cell line. Crude exosomes obtained by ultracentrifugation were fractionated by density gradient centrifugation into ten fractions each having an equal volume with the fraction on the top of the tube (having the lowest density) designated as fraction 1. For proteins expressed in each fraction which was confirmed by Western blotting, the number of particles in each fraction was analyzed with nanoparticle analyzer (NanoSight LM 10, manufactured by Malvern Instruments). FIG. 5, left shows the results of analyzing a cell lysate (C. L.), a crude microvesicle fraction (MV 10K), and each fraction with anti-CD63 antibody or an anti-CD81 antibody by Western blotting. As shown in FIG. 5, right, the number of particles is large in fractions 1 and 3, but CD63 and CD81 commonly used as exosome marker proteins are particularly highly expressed in fractions 3 and 4 (FIG. 5, left). It is therefore thought that exosomes with CD63 and CD81 on the surface are concentrated in fractions 3 and 4.

Each of the above-mentioned exosome fractions 1, 2, 3, 7 and 8 was mixed with M40 in PBS buffer (10 mM Na₂HPO₄, 2 mM KH₂PO₄, 137 mM NaCl, 2.7 mM KCl, pH 7.4) containing 0.01% BSA and were allowed to stand at room temperature for 30 minutes. For comparison, a sample in which PBS alone instead of exosomes and M40 were mixed, or a sample in which BSA-modified gold nanoparticles (hereinafter referred to as B40) and fraction 3 in which exosome particles having CD63 and CD81 exposed on its surface were concentrated was prepared in the same manner. Each solution was prepared so that the number of particles contained in the exosome fraction was 4.4×10¹⁰, and approximately 3.6 times more gold nanoparticles than the particles were added thereto.

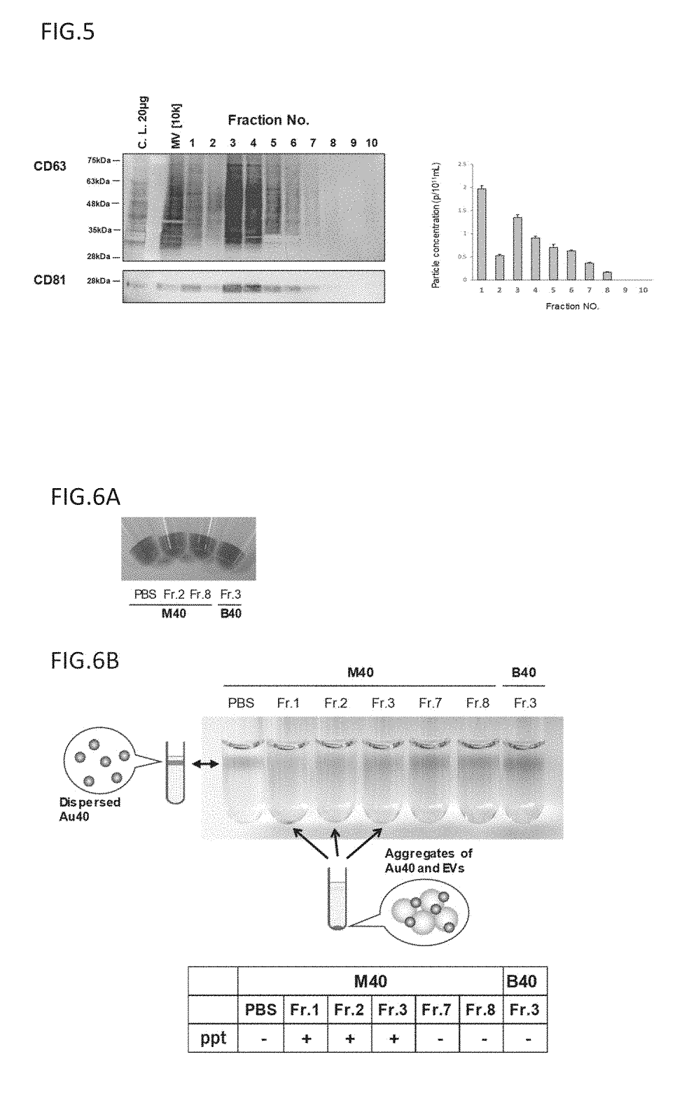

Each of the solutions that were allowed to stand at room temperature for 30 minutes exhibited a red color derived from gold nanoparticles, but no noticeable color difference was observed depending on the fractions or the presence or absence of exosomes (FIG. 6A).

The mixture of the exosome of each fraction or PBS and M40, or the mixture of the exosome of fraction 3 and B40 was layered on an iodixanol density gradient solution, and subjected to low-speed centrifugation. Specifically, 20 mM HEPES (pH 7.4) solutions containing 14%, 12% and 10% iodixanol were prepared, they are layered in a glass tube (Micro tube No. 1, manufactured by Maruemu Corporation) in the following order from the bottom: 200 μL of a 14% iodixanol solution (density: 1.079 g/mL), 100 μL of a 12% iodixanol solution (density: 1.069 g/mL) and 100 μL of a 10% iodixanol solution (density: 1.058 g/mL) to prepare a density gradient solution with three density layers. The mixture of the exosomes of each fraction or PBS and M40, or the mixture of the exosomes of fraction 3 and B40 (each 40 μL) was layered on the density gradient solution, and subjected to centrifugation at 1,500 g for five minutes in a centrifuge (centrifuge: H-3R, rotor: RF-110, both manufactured by KOKUSAN Co. Ltd.) set at 4° C.

A photograph of the tube after centrifugation and a schematic view illustrating the state in which gold nanoparticles and exosomes were present are shown in FIG. 6B. For the mixture with M40, no precipitate was observed in the exosome-free sample (containing PBS only) and in the exosome mixture of each of fractions 7 and 8, but a red-colored precipitate was observed in the exosome mixture of each of fractions 1, 2 and 3. On the other hand, no precipitate was observed in the mixture of B40 and the exosome of fraction 3. It is thought that MFGE8 conjugated with a gold nanoparticle has interacted with an exosome subclass having a large number of phosphatidylserine exposed thereon to produce a precipitate.

It was determined in the following procedure that the amount of phosphatidylserine exposed on the exosome surface was more in fraction 1 than in fraction 3. Using annexin V the most commonly used for the detection of phosphatidylserine on the surface of a cell membrane, phosphatidylserine on the surface of an exosome membrane was labelled with a gold particle, and was observed with an atomic force microscope (AFM).

Specifically, a drop of each of exosome fractions 1 and 3 was put on an aminated mica substrate and allowed to stand for one hour so as to adsorb the exosome on the surface of the substrate. After washing the substrate with PBS, a PBS solution containing 5% BSA was put on the substrate and allowed to stand at room temperature for five minutes so as to perform blocking, and washed once with PBS. Biotinylated annexin V was added thereto and allowed to stand at room temperature for two hours for binding and washed with PBS. Thereafter, an anti-biotin antibody conjugated with a 20 nm gold particle was added as a secondary antibody thereto and allowed to stand overnight at 4° C. After washing with PBS, it was subjected to fixation with glutaraldehyde and osmium tetroxide. It was washed three times with PBS and five times with water, allowed to stand at room temperature for drying, and observed in the air at room temperature with AFM (Asylum MFP-3D, manufactured by Oxford Instruments).

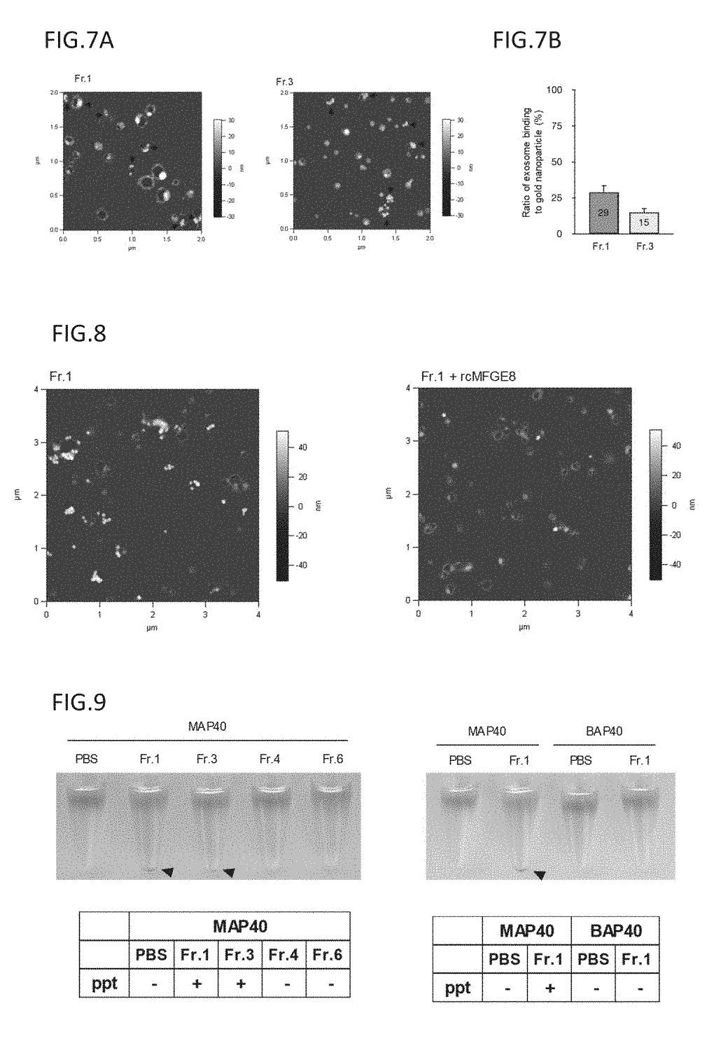

AFM images of fractions 1 and 3 are shown in FIG. 7A. Arrows indicate gold particles bound via annexin V. The number of exosomes having gold particles bound thereto was measured. The proportion of exosomes having gold particles bound thereto was 29% in fraction 1 and 15% in fraction 3 (FIG. 7B). From this, it is thought that exosomes having phosphatidylserine exposed thereon are more in fraction 1 than in fraction 3.

It was also confirmed by AFM observation that M40 binds to a MiaPaca-2 exosome. Instead of using annexin V and anti-biotin antibody, M40 in PBS solution containing 1% BSA was put and allowed to stand at room temperature for two hours. It was washed three times with PBS and five times with water, and was observed with AFM in the same manner as above. As shown in FIG. 8, it was observed that a large number of M40 were bound to the exosome on the substrate (FIG. 8, left). Adding recombinant MFGE8 (2767-MF, manufactured by R & D Systems) greatly decreased the number of M40 bound to the exosome (FIG. 8, right). From this, it is thought that the binding of M40 to the exosome depended on MFGE8 at least in this Example.

### Example 5

### Detection of Exosome Using MFGE8-Modified Alloy Nanoparticle

Using the 40 nm alloy nanoparticle comprising platinum and gold modified with MFGE8 (hereinafter referred to as MAP40) prepared in Example 2, an exosome was detected utilizing the binding between MFGE8 and phosphatidylserine on the surface of the exosome.

Each of exosome fractions 1, 3, 4 and 6 fractionated from a culture medium of MiaPaca-2 in the same manner as above was mixed with MAP40 in PBS containing 0.1% BSA, and was allowed to stand at room temperature for 30 minutes. For comparison, a sample in which PBS alone instead of exosomes and MAP40 were mixed, or a sample in which BSA-modified alloy nanoparticles (hereinafter referred to as BAP40) and fraction 1 were mixed was prepared in the same manner. The number of particles contained in the exosome fraction was 4×10¹⁰ in each solution, and a certain amount of alloy particles (having a concentration at which the absorbance at 400 nm was 2.4) was added thereto so that the ratio of the number of alloy particles to the number of particles contained in each exosome fraction was the same.

Twenty μL of the mixture of the exosomes and MAP40 after allowed to stand was layered on a density gradient solution containing 40 μL of 10% iodixanol overlaid on 100 μL of 14% iodixanol and subjected to low-speed centrifugation. The centrifugation was performed at 600 g for five minutes in a centrifuge (centrifuge: Model 5500, rotor: ST-722M, both manufactured by KUBOTA CORPORATION) set at 20° C.

Photographs of the tube after centrifugation are shown in FIG. 9. For the mixtures with MAP40, in the exosome-free sample (containing PBS only) and in the mixture with each of exosome fractions 4 and 6, black-colored bands derived from the alloy particles was observed on the upper portion thereof but no precipitate was observed. However, in the mixture of MAP40 and each of fractions 1 and 3, the black-colored band became lighter, and a black precipitate was observed. On the other hand, no precipitate was observed in the mixture of BAP40 and fraction 1. As described above, for the mixture with MAP40, a precipitate was observed in the exosome-containing sample similarly to the mixture with MA40.

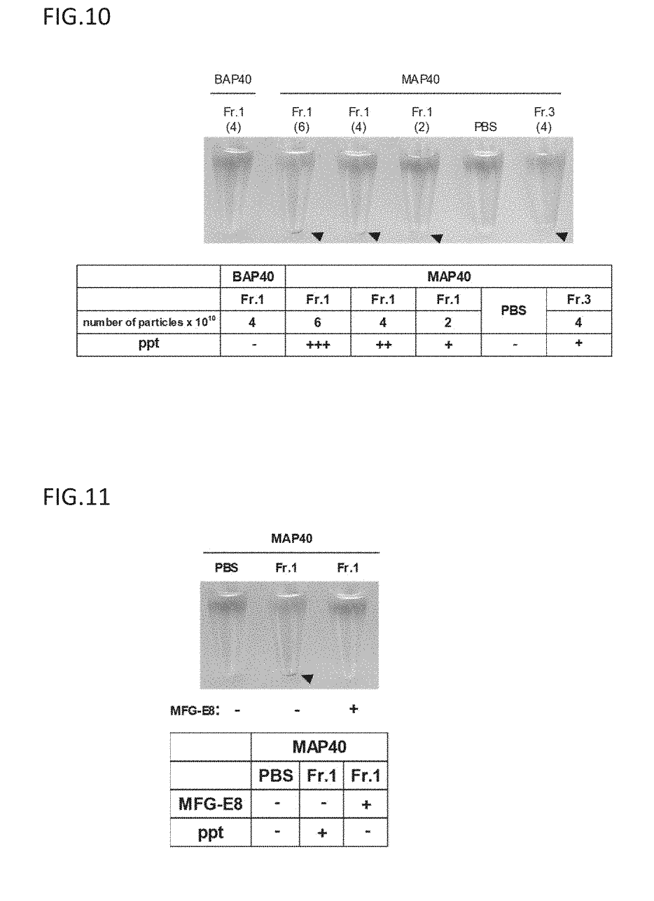

The amount of precipitate varied depending on the number of exosome particles contained in the samples (FIG. 10). Centrifugation was performed under the same conditions as above, except that the number of exosome particles contained in fraction 1 was changed to 2, 4 or 6×10¹⁰ particles. In FIG. 10, the number in parenthesis indicates the number of exosome particles (×10¹⁰). In comparison after changing the number of exosome particles contained in fraction 1 to 2, 4 or 6×10¹⁰, the more the number of exosome particles was, the more the precipitate amount also was.

It was then confirmed by adding MFGE8 to the samples that the precipitation was derived from the specific binding of molecules on the exosome surface and nanoparticles. MFGE8 (0.56 μM) was mixed with fraction 1 and allowed to stand at room temperature for 40 minutes, and then subjected to mixing with MAP40 and centrifugation in the same manner as above. When fraction 1 and MFGE8 were mixed before mixing with MAP40, no precipitate was observed (FIG. 11).

### Example 6

### Detection of Exosome Using Anti-CD81 Antibody-Modified Gold Nanoparticle

Exosomes were detected using gold nanoparticles prepared in Example 3 (hereinafter referred to as 81A40).

Exosome samples were prepared in the same manner as in Example 4, and each of exosome fractions 1, 3, 4 and 6 obtained by fractionation was mixed with 81A40 in PBS containing 0.1% BSA, and the mixture was allowed to stand at room temperature for two hours. For comparison, a sample in which PBS alone and 81A40 were mixed, or a sample in which gold nanoparticles modified with an isotype control antibody (hereinafter referred to as ctA40) and fraction 3 were mixed was prepared in the same manner. Each solution was prepared so that the number of particles contained in the exosome fraction was 3.3×10¹⁰, and approximately 4.9 times more gold nanoparticles than the particles were added thereto.

The mixture of the exosomes and the gold nanoparticles after allowed to stand was layered on a density gradient solution of iodixanol and subjected to low-speed centrifugation. Specifically, 20 mM HEPES (pH 7.4) solutions containing 14% and 10% iodixanol were prepared, they are layered in a 0.2 mL plastic tube (a PCR tube manufactured by Greiner Bio-One International GmbH) in the following order from the bottom: 100 μL of 14% iodixanol solution and 40 μL of 10% iodixanol solution to prepare two layers of density gradient. Twenty μL of the mixture of the exosomes of each fraction and gold nanoparticles was layered on the density gradient, and subjected to centrifugation at 1,000 g for five minutes in a centrifuge (centrifuge: Model 5500, rotor: ST-722M, both manufactured by KUBOTA CORPORATION) set at 20° C.

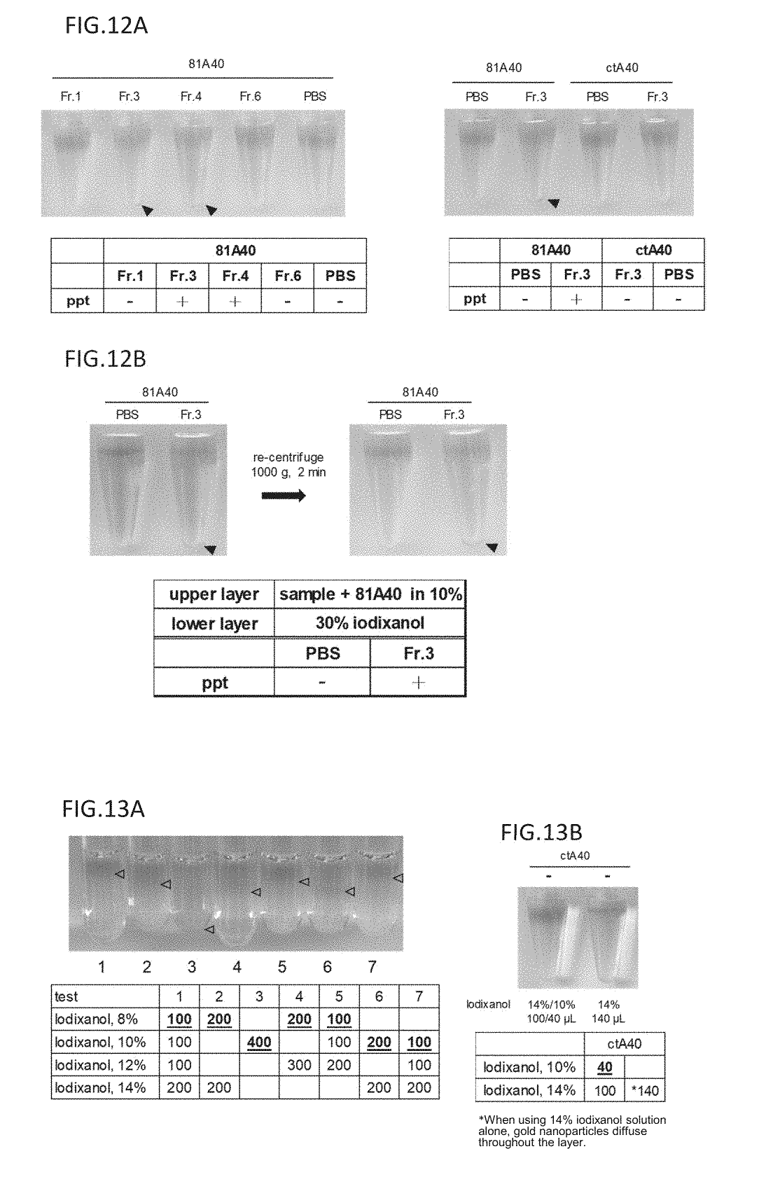

Photographs of the tube after centrifugation are shown in FIG. 12A. For the mixtures with 81A40, a gold particle coated with anti-CD81 antibody, red-colored bands derived from the gold nanoparticles was observed on the upper portion but no precipitate was observed in the exosome-free sample (PBS only) and in the mixture containing each of exosome fractions 1 and 6 obtained by fractionation. However, in the mixture of 81A40 and each of fractions 3 and 4, the upper red-colored band was lighter, and a red-colored precipitate was observed. On the other hand, no precipitate was observed in the mixture of ctA40 and the exosome of fraction 3. As shown in FIG. 5, CD81 is highly expressed on exosome in fractions 3 and 4. It is therefore thought that 81A40 interacts with an exosome subclass capable of highly expressing CD81, to produce a precipitate, in this Example.

Next, it will be shown that even when a sample is layered on a single density layer, exosomes can be detected. In the same manner as in Example 4, the exosome sample was prepared from MiaPaca-2, and the exosome fraction 3 obtained by fractionation or PBS was mixed with 81A40. Twenty μL of the mixed solution was mixed with 20 μL of 20% iodixanol (iodixanol final concentration: 10%) and allowed to stand at room temperature, and then overlaid on 160 μL of 30% iodixanol.

The centrifugation was performed at 1,000 g for five minutes in a centrifuge (centrifuge: Model 5500, rotor: ST-722M, both manufactured by KUBOTA CORPORATION) set at 20° C. As shown in FIG. 12B, left, exosome precipitates were detected in the sample in which fraction 3 and 81A40 were mixed. In addition, by another centrifugation at 1,000 g for two minutes, the precipitates were able to be clearly confirmed in the sample having fraction 3 mixed therein. Even when the sample solution in which the exosomes and the modified nanoparticles were mixed was layered on a single density layer, the exosomes were able to be precipitated and detected.

### Example 7

### Study of Density

Changing the density of a medium, a study was performed for the density at which extracellular vesicles bound to nanoparticles could be fractionated. Using BSA-modified gold nanoparticles, the density of the medium in which the gold nanoparticles did not precipitate was studied. As shown in the table of FIG. 13A, in the Tests 1 to 7, 100 to 400 μL of 8%, 10%, 12% and 14% iodixanol were layered, and 40 μL of a sample solution in which gold nanoparticles were mixed with PBS was layered thereon and centrifuged at 1,000 g for 10 minutes at 4° C. with a centrifuge manufactured by KOKUSAN Co. Ltd.

The presence of the gold nanoparticles is indicated by arrows in FIG. 13A and underlined in the table. In any of the tests, gold nanoparticles do not exist in media having 12% or more of iodixanol after centrifugation. As shown in Tests 1 and 5, even in the case of 10% iodixanol, when a medium with a concentration by 2% lower than that is layered, the gold nanoparticles do not enter the 10% iodixanol layer. In such a single layer as in Test 3, the nanoparticles diffuse downward. Although no data is shown, even in the case of 12% or 14% iodixanol, when a single layer was used, diffusion of nanoparticles was observed.

In addition, the density gradient was studied. Twenty μL of ctA40 alone was layered on a density gradient solution with graded two densities obtained by layering iodixanol solutions in a tube in the following order from the tube bottom: 100 μL of 14% iodixanol and 40 μL of 10% iodixanol, or on 140 μL of 14% iodixanol alone, and subjected to centrifugation at 1,000 g for five minutes in the same manner as above (centrifuge: Model 5500, rotor: ST-722M, both manufactured by KUBOTA CORPORATION) (FIG. 13B). When using the 14% iodixanol solution alone, gold nanoparticles diffuse, whereas when using the step gradient, the gold nanoparticles do not precipitate, in this Example.

These results show that, in this Example, in a single layer, the nanoparticles diffuse, thus making it more challenging to separate them, but by providing two or more layers, heavy complexes of nanoparticles and exosomes formed by their binding can be separated. Accordingly, it was concluded that at least two layers with different densities, including a sample layer, can be used for precipitating exosomes, in some embodiments.

The above results show that, when a solution containing extracellular vesicles and nanoparticles modified with a ligand that binds to a molecule on the surface of the extracellular vesicles is prepared, so that the concentration of an iodixanol solution is 10%, that is, its density is 1.053 g, and layered on a medium with the higher density, the nanoparticles bound to the extracellular vesicles precipitate but the nanoparticles not bound thereto do not precipitate, and the extracellular vesicles can be fractionated depending on their surface molecules.

### Example 8

### Study Using Crude Exosome

If exosomes can be detected without purification, it will be very useful in clinical examinations and the like. Accordingly, a detection study was performed in the same manner using a crude exosome fraction.

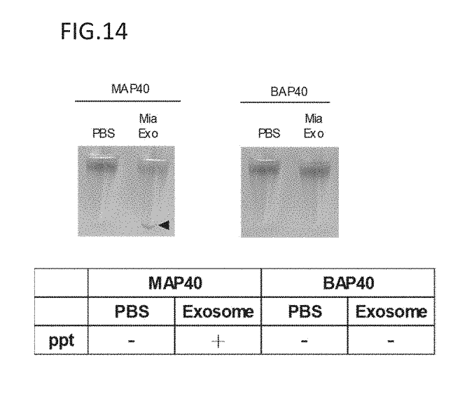

Twenty μL of a sample in which crude exosomes from MiaPaca-2 concentrated by ultracentrifugation and MAP40 or BAP40 were mixed was layered on density gradient layers in the following order from the tube bottom: 100 μL of 14% iodixanol, 40 μL of 10% iodixanol, and subjected to centrifugation at 800 g for five minutes at 4° C. with a centrifuge manufactured by KOKUSAN Co. Ltd. (FIG. 14). Precipitation was observed in the sample in which MAP40 and crude exosomes were mixed, whereas precipitation was not observed in the sample in which BAP40 and crude exosomes were mixed.

Even in the case of not using any purified sample, this method can be used to separate exosomes of a particular subclass and is very useful method for examination or research.

As shown in the above Examples, a nanoparticle coated with a ligand that binds to a molecule present on the surface of exosomes can be used to precipitate the exosomes by low-speed centrifugation, for example, in the range of 500 g to 1,500 g. Since exosomes can be classified into subclasses utilizing molecules present on the surface thereof, they can be classified into subclasses and thereafter subjected to analysis of exosomes.

Example, non-limiting experimental data are included in this specification to illustrate results achievable by various embodiments of the systems and methods described herein. All ranges of data and all values within such ranges of data that are shown in the figures or described in the specification are expressly included in this disclosure. The example experiments, experimental data, tables, graphs, plots, figures, and processing and/or operating parameters (e.g., values and/or ranges) described herein are intended to be illustrative of operating conditions of the disclosed systems and methods and are not intended to limit the scope of the operating conditions for various embodiments of the methods and systems disclosed herein. Additionally, the experiments, experimental data, calculated data, tables, graphs, plots, figures, and other data disclosed herein demonstrate various regimes in which embodiments of the disclosed systems and methods may operate effectively to produce one or more desired results. Such operating regimes and desired results are not limited solely to specific values of operating parameters, conditions, or results shown, for example, in a table, graph, plot, or figure, but also include suitable ranges including or spanning these specific values. Accordingly, the values disclosed herein include the range of values between any of the values listed or shown in the tables, graphs, plots, figures, etc. Additionally, the values disclosed herein include the range of values above or below any of the values listed or shown in the tables, graphs, plots, figures, etc. as might be demonstrated by other values listed or shown in the tables, graphs, plots, figures, etc. Also, although the data disclosed herein may establish one or more effective operating ranges and/or one or more desired results for certain embodiments, it is to be understood that not every embodiment need be operable in each such operating range or need produce each such desired result. Further, other embodiments of the disclosed systems and methods may operate in other operating regimes and/or produce other results than shown and described with reference to the example experiments, experimental data, tables, graphs, plots, figures, and other data herein. Thus, the invention has been described in several non-limiting embodiments. It is to be understood that the embodiments are not mutually exclusive, and elements described in connection with one embodiment may be combined with, rearranged, or eliminated from other embodiments in suitable ways to accomplish desired design objectives. No single feature or group of features is necessary or required for each embodiment. All possible combinations and sub-combinations of elements are included within the scope of this disclosure.
For purposes of summarizing the present invention, certain aspects, advantages and novel features of the present invention are described herein. It is to be understood, however, that not necessarily all such advantages may be achieved in accordance with any particular embodiment. Thus, the present invention may be embodied or carried out in a manner that achieves one or more advantages without necessarily achieving other advantages as may be taught or suggested herein.
As used herein any reference to “one embodiment” or “some embodiments” or “an embodiment” means that a particular element, feature, structure, or characteristic described in connection with the embodiment is included in at least one embodiment. The appearances of the phrase “in one embodiment” in various places in the specification are not necessarily all referring to the same embodiment. Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. In addition, the articles “a” or “an” or “the” as used in this application and the appended claims are to be construed to mean “one or more” or “at least one” unless specified otherwise.
As used herein, the terms “comprises,” “comprising,” “includes,” “including,” “has,” “having” or any other variation thereof, are open-ended terms and intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), or both A and B are true (or present). As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present.
Thus, while only certain embodiments have been specifically described herein, it will be apparent that numerous modifications may be made thereto without departing from the spirit and scope of the invention. Further, acronyms are used merely to enhance the readability of the specification and claims. It should be noted that these acronyms are not intended to lessen the generality of the terms used and they should not be construed to restrict the scope of the claims to the embodiments described therein.

What is claimed is:

1. A method for detecting, fractionating and/or purifying extracellular vesicles, comprising:
preparing nanoparticles coated with a ligand that specifically binds to a molecule present on the surface of the extracellular vesicles to be detected, and a sample solution in which the nanoparticles and the extracellular vesicles are mixed;
providing at least one medium layer with a density higher than that of the sample solution;
overlaying the sample solution on the at least one medium layer; and
subjecting the at least one medium layer overlaid with the sample solution to low-speed centrifugation.

2. The method according to claim 1, wherein the centrifugation comprises step density gradient centrifugation or continuous density gradient centrifugation.

3. The method according to claim 1, wherein the extracellular vesicles comprise exosomes.

4. The method according to claim 2, wherein the extracellular vesicles comprise exosomes.

5. The method according to claim 1, wherein the nanoparticles comprise particles containing a sixth periodic element of the periodic table.

6. The method according to claim 2, wherein the nanoparticles comprise particles containing a sixth periodic element of the periodic table.

7. The method according to claim 3, wherein the nanoparticles comprise particles containing a sixth periodic element of the periodic table.

8. A kit for detecting, classifying and/or purifying extracellular vesicles, comprising:
nanoparticles coated with a ligand that specifically binds to a molecule present on the surface of the extracellular vesicles to be detected; and
a density gradient solution.

9. The kit according to claim 8, wherein the extracellular vesicles comprise exosomes.
